(19) United States
(12) Patent Application Publication
(10) Pub. No.: US 2010/0249541 A1
(43) Pub. Date: Sep. 30, 2010

(54) Methods and Apparatus for Processing Physiological Data Acquired from an Ambulatory Physiological Monitoring Unit

(75) Inventors: Yacov GEVA, Chicago, IL (US); Joseph Clauser, Chicago, IL (US); Veronica Newberg, Midlothian, IL (US); Michael Jandes, Round Lake, IL (US); Marikay Menard, Downers Grove, IL (US); George Michelson, Glenview, IL (US); Melissa Petrucci, Wauconda, IL (US); Jason Van Schagen, Streamwood, IL (US)

Correspondence Address: PROSKAUER ROSE LLP, ONE INTERNATIONAL PLACE, BOSTON, MA, 02110, US

(73) Assignee: LifeWatch Corp., Rosemont, IL (US)

(21) Appl. No.: 12/731,334

(22) Filed: Mar. 25, 2010

Related U.S. Application Data
(60) Provisional application No. 61/164,318, filed on Mar. 27, 2009.

(51) Int. Cl. A61B 5/00

(52) U.S. Cl. 600/301

(57) ABSTRACT

A physiologic monitoring system and corresponding methods provide rapid and detailed analysis of data for one or more physiologic parameters to achieve a quick and accurate medical diagnosis. An ambulatory physiological monitoring unit acquires physiologic data, automatically analyzes it to detect an event, and transmits information regarding the event and physiologic data associated with the event across a communications network to a monitoring center, where the event information is analyzed and triaged. The monitoring center can also perform a retrospective analysis based on the physiological data associated with the event to provide an in-depth analysis of the detected event and an accurate diagnosis. The monitoring center can also request additional or different physiological data to refine the analysis. As a result, the physiological monitoring system and corresponding methods can ensure that timely and appropriate intervention is taken to reduce a patient's discomfort, pain, injury, or risk of death.

### CROSS-REFERENCE TO RELATED APPLICATION

This application claims priority to and the benefit of U.S. Provisional Patent Application Ser. No. 61/164,318, entitled “Methods and Apparatus for Processing Physiological Data Acquired from an Ambulatory Physiological Monitoring Unit,” and filed on Mar. 27, 2009, the entire contents of which is incorporated herein by reference.

### FIELD OF THE INVENTION

The present invention pertains to methods and apparatus for accurately monitoring and evaluating the health of a person using an ambulatory physiological monitoring unit and a monitoring center, which communicate with each other across a network.

### BACKGROUND OF THE INVENTION

Many ambulatory health monitors have been developed by medical technology companies and used by medical professionals to monitor and diagnose the health of their patients. Many of these ambulatory health monitors are used to record electrocardiography (ECG) data. A patient wears an ambulatory ECG monitor for long periods of time ranging from many hours to many days. After this long period of time, the monitor is returned to the medical professional for detailed analysis of the ECG data, such as a Holter analysis of the ECG data. The medical professional uploads the ECG data to a computer, which executes software for performing a detailed analysis of the ECG data and displays the results on a screen that can be reviewed by a medical professional. Although the results of the Holter analysis are detailed and accurate, by the time the medical professional diagnoses the patient, the patient may have already suffered from a severe heart-related injury.

Advances in wireless, networking, and electronics technologies have allowed medical technology companies to develop and deploy wireless ambulatory physiological monitoring units so that information regarding a physiologic parameter can be transmitted to a remote monitoring center. Some wireless ambulatory physiological monitoring units acquire ECG data and transmit a small portion or a representation of this data to a remote monitoring center via a cellular network. This information may be current and helpful, but it may be insufficient to arrive at an accurate and conclusive diagnosis of the patient's physiological condition.

### SUMMARY OF THE INVENTION

More timely and accurate information regarding the physiological condition of a patient can be obtained by combining real-time analysis of physiological data in an ambulatory physiological monitoring unit with the detailed retrospective analysis of the physiological data at a remote monitoring center. The invention in one aspect features a method for monitoring the patient, which includes the steps of acquiring physiological data, analyzing segments of physiological data and generating real-time analysis data for each segment, transmitting real-time analysis data to a remote monitoring center, and transmitting physiological data that is associated with the real-time analysis data to the remote monitoring center.

Analyzing segments of physiological data includes monitoring for a physiological condition or event. If the physiological condition or event is detected, real-time analysis data is generated to include information regarding the physiological condition or event. Detecting a physiological condition or event can include determining whether the physiological data or processed physiological data reaches a predetermined level.

In some embodiments, the physiological data includes data for any number of physiologic parameters of any body system, such as electrocardiography or pulse-oximetry data. The real-time analysis data can be transmitted to the remote monitoring center at predetermined intervals, or in response to input from a human user, an algorithm, or both. The physiological data can be transmitted to the remote monitoring center by storing the physiological data in a memory module of an ambulatory physiological monitoring unit and by uploading the physiological data from the memory module to the remote monitoring center. In some embodiments, the data acquisition module is in wireless communications with the real-time analysis module and the memory module.

In some embodiments, the method may further include detecting a message requesting physiological data or real-time analysis data for at least one physiologic parameter, and executing the data acquisition module and real-time analysis module to acquire and to analyze physiological data for at least one physiologic parameter. A user interface associated with the ambulatory physiological monitoring unit can generate and transmit the message.

Another aspect of the invention features a method for monitoring the patient, which includes the steps of receiving real-time analysis data, which is based on an analysis of segments of physiological data, from a remote ambulatory physiological monitoring unit, receiving physiological data associated with the real-time analysis data from the remote ambulatory physiological monitoring unit, and invoking an analysis module to generate detailed retrospective analysis based on the physiological data.

In some embodiments, the real-time analysis data is information regarding a physiological event or condition detected by the remote ambulatory physiological monitoring unit. The physiological data can include data for a plurality of physiologic parameters associated with the same and/or different body systems. In some embodiments, the retrospective analysis module correlates physiological data for the plurality of physiologic parameters.

The real-time analysis data can be received via a wireless communications link and the physiological data can be uploaded from a memory module on the remote ambulatory physiological monitoring unit to the monitoring center via a wired communications link.

In some embodiments, an evaluation module evaluates the real-time analysis data or retrospective analysis data to determine the physiological status of the patient. The evaluation module can include a user interface module for allowing a user (e.g., a medical professional) to interact with the real-time analysis data and retrospective analysis data. For example, if the user interface module detects a user input command requesting physiological data or real-time analysis data, a message requesting physiological data or real-time analysis data can be transmitted to the remote ambulatory monitoring unit. The evaluation module can also automatically determine whether the physiological data and real-time analysis data is sufficient to generate conclusive diagnostic information.

The messages transmitted to the ambulatory physiological monitoring unit can include a request for additional physiological data or a request for physiological data for a different physiologic parameter. These messages can also include information regarding the physiological status of the patient.

Another aspect of the invention features an ambulatory physiological monitoring unit. In some embodiments that ambulatory physiological monitoring unit includes a physiological data acquisition module for acquiring physiological data; a real-time analysis module, which analyzes segments of the physiological data and generates real-time analysis data; a storage module for storing the physiological data; and a transceiver for transmitting the real-time analysis data to a remote monitoring center via a communications network at predetermined intervals or in response to at least one event or physiological condition detected by the real-time analysis module. The transceiver can also transmit physiological data associated with the real-time analysis data to the remote monitoring center for retrospective analysis of the physiological data.

In some embodiments, the ambulatory physiological monitoring unit can include a user interface module, which accepts input from a user regarding at least one event or physiological condition and generates a message containing information regarding the event or physiological condition. The transceiver can then transmit the message to the remote monitoring center.

Another aspect of the invention features a system for monitoring a patient. The system can include an ambulatory physiological monitoring unit and a remote monitoring center, which is in communication with the ambulatory physiological monitoring unit via a network. The ambulatory physiological monitoring unit can include a physiological data acquisition module for acquiring physiological data; a real-time analysis module for analyzing segments of the physiological data and for generating real-time analysis data for each segment of physiological data; a storage module for storing the physiological data; and a transceiver for transmitting the real-time analysis data and physiological data associated with the real-time analysis data via a communications network. The remote monitoring center can further include a transceiver for receiving the real-time analysis data and the physiological data from the ambulatory physiological monitoring unit and for transmitting messages requesting physiological data to the ambulatory physiological monitoring unit via the communications network; a retrospective analysis module for performing a retrospective analysis based on the physiological data and for generating retrospective analysis data; and an evaluation module for evaluating the physiological condition of the patient based on the real-time analysis data or the retrospective analysis data.

The foregoing and other objects, aspects, features, and advantages of the invention will become more apparent from the following description and from the claims.

### BRIEF DESCRIPTION OF THE DRAWINGS

The foregoing and other objects, features, and advantages of the invention, as well as the invention itself, will be more fully understood from the following illustrative description, when read together with the accompanying drawings which are not necessarily to scale.

FIG. 1 is a block diagram of a physiological monitoring system according to embodiments of the present invention.

FIG. 2 is a dataflow diagram of a physiological monitoring system according to an embodiment of the present invention.

FIG. 3A is a block diagram of an ambulatory physiological monitoring unit according to embodiments of the present invention.

FIG. 3B is a block diagram of a remote monitoring center according to embodiments of the present invention.

FIG. 4 is a block diagram of an ambulatory physiological monitoring unit, that comprises a sensor module and a monitor according to embodiments of the present invention.

FIGS. 5A and 5B are functional block diagrams of a physiological monitoring system that includes an ambulatory physiological monitoring unit having a sensor module and a monitor, a web service computer, and a clinical system computer in accordance with embodiments of the present invention.

FIG. 6 is a diagram of an ambulatory physiological monitoring system that includes an ambulatory health monitoring unit in accordance with embodiments of the present invention.

FIG. 7 is a block diagram of an ambulatory physiological monitoring unit in accordance with embodiments of the present invention.

FIG. 8 is a data flow diagram showing the acquisition, storage, and real-time analysis of physiological data in accordance with an embodiment of the present invention.

FIG. 9 is a data flow diagram showing the retrospective analysis of the physiological data that has been received from an ambulatory physiological monitoring unit in accordance with an embodiment of the present invention.

FIGS. 10-12 are flowcharts of processes which can be executed on an ambulatory physiological monitoring unit in accordance with embodiments of the present invention.

FIGS. 13-15 are flowcharts of processes which can be executed on a remote monitoring center in accordance with embodiments of the present invention.

### DETAILED DESCRIPTION OF ILLUSTRATIVE EMBODIMENTS

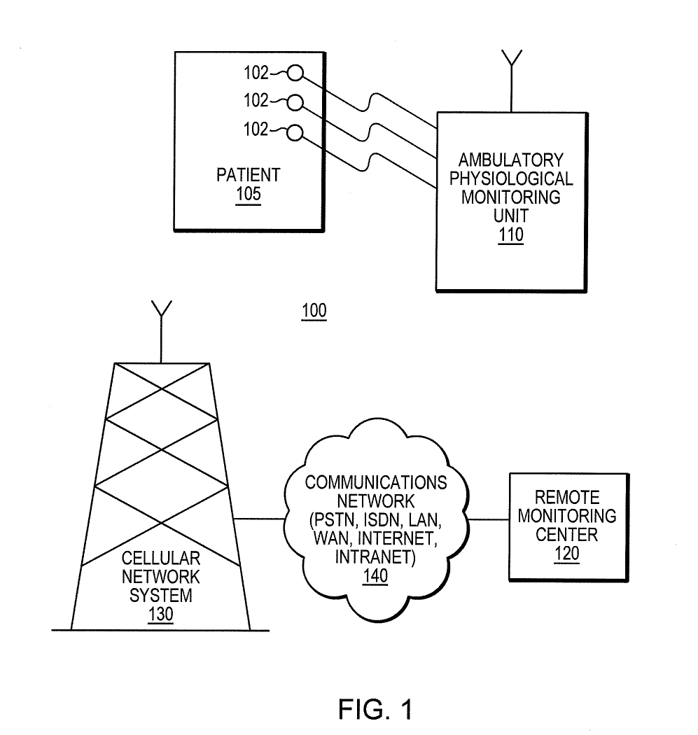

FIG. 1 shows a diagram of a physiological monitoring system 100 according to embodiments of the present invention. An ambulatory physiological monitoring unit 110 (e.g., an ambulatory cardiac monitor) connects to a patient 105 through sensors 102 (e.g., electrodes), which measure a physiologic parameter of a body system. The ambulatory physiological monitoring unit 110 may communicate with a remote monitoring center 120 via a cellular network system 130 and a communications network 140, such as a public switched telephone network (PSTN), ISBN, LAN, WAN, Internet, or intranet. The remote monitoring center 120 may be located at an independent call center, hospital, or doctor's office, where a medical professional(s) may be notified of an abnormal physiological condition (e.g., an abnormal arrhythmia) that is detected by the ambulatory physiological monitoring unit 110. The remote monitoring center 120 may itself communicate via a communications network with other electronic devices such as a personal digital assistant or BlackBerry® device to provide information on the physiological status of the patient 105 to interested individuals, such as a medical professional(s).

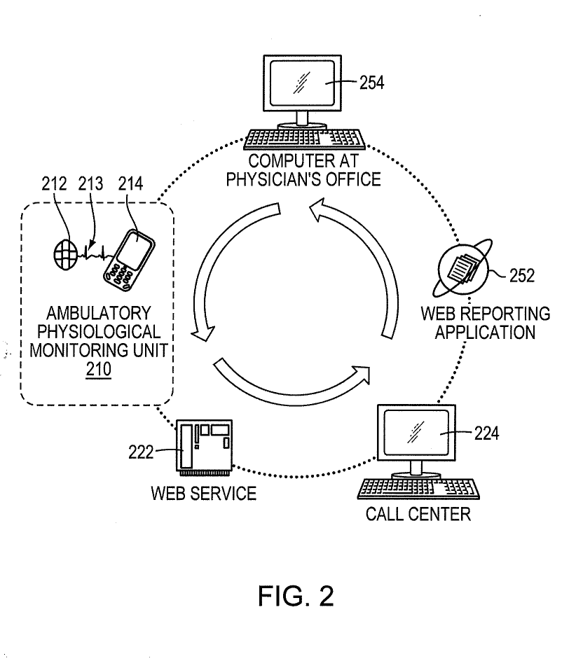

FIG. 2 is a diagram that illustrates the different electronic devices and computers that interact in the ambulatory physiological monitoring system 100 of FIG. 1, according to embodiments of the present invention. As illustrated in FIG. 2, a ambulatory physiological monitoring unit 210 includes a physiological sensor module 212 (e.g., a cardiac sensor), which may also be referred to as a sensor module or a physiological data acquisition module, and an ambulatory physiological monitor 214 (e.g., an ambulatory cardiac monitor). The physiological sensor module 212 transmits physiological data 213 (e.g., electrocardiography data) to the ambulatory physiological monitor 214. The ambulatory physiological monitor 214 processes the physiological data and transmits a message containing a portion of the physiological data 213 and/or information regarding the processed physiological data to a database or other Web service 222, which stores and maintains the messages.

A computer located at a call center 224 may execute a web application, which allows an attendant, technician, or clinician to access or receive notifications of the messages. Like the computer at the call center 224, a computer at a physician's office 254 may also access or receive notifications of the messages through a Web reporting application 252. The computer at the physician's office 254 can also communicate with the ambulatory physiological monitoring unit 210. For example, a physician using the computer 254 can send a message to the ambulatory physiological monitor 210 giving instructions or information to the user of the ambulatory physiological monitoring unit 210. A physician using the computer 254 can also remotely program or change parameters in the sensor module 212 or the ambulatory physiological monitor 214.

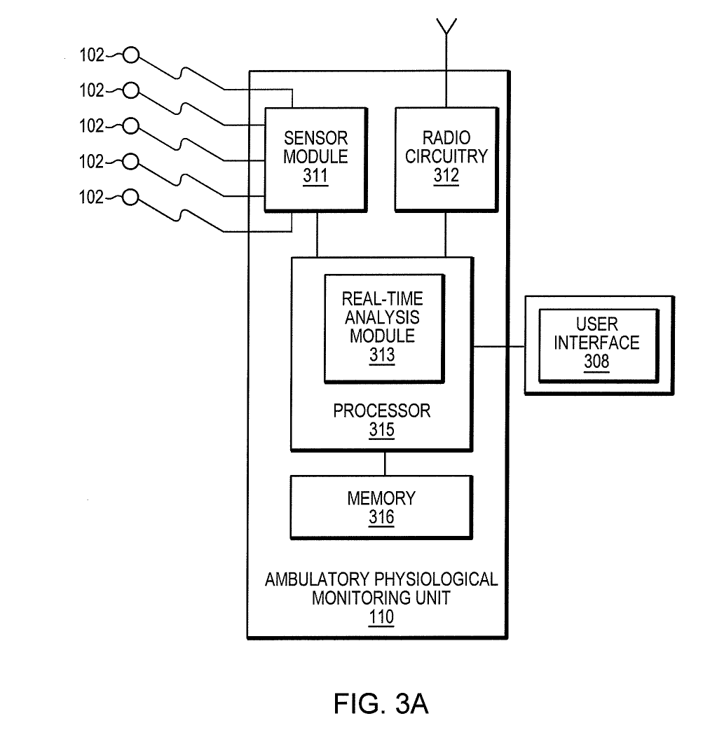

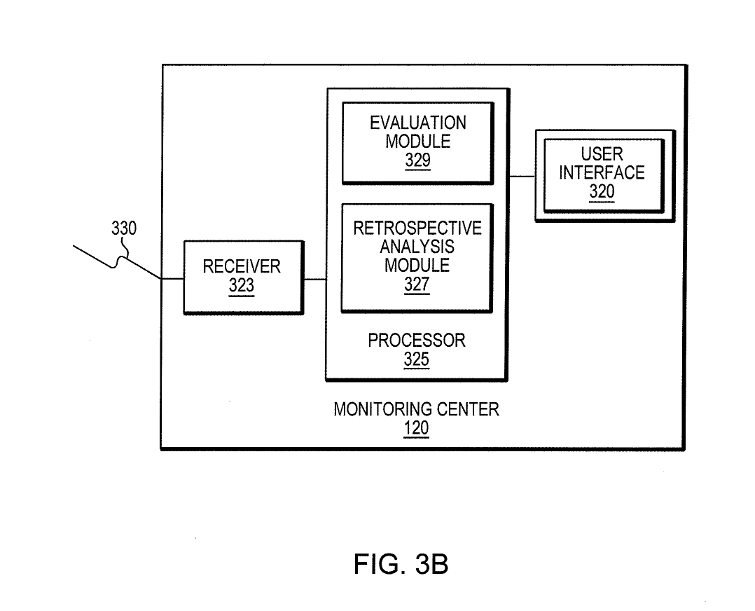

FIG. 3A illustrates an ambulatory physiological monitoring unit 110 according to an embodiment of the present invention. The ambulatory physiological monitoring unit 110 may include a sensor module 311, radio circuitry 312, a processor 315, and memory 316. The sensor module 311 may include a series of ports to which a series of sensors 102 may connect. The sensor module 311 acquires physiological data from the series of sensors 102, converts the data into digital form, and provides a digital physiological data stream to the processor 315. The processor 315 executes a real-time analysis module 313, which analyzes segments of the digital physiological data stream in real time or in near real-time. The processor 315 also stores the digital physiological data in the memory 316. The radio circuitry 312 can transmit real-time analysis data (e.g., information on the results of the real-time analysis of the digital physiological data stream, including whether a physiological event has been detected) and a portion of the digital physiological data stream associated with the real-time analysis data.

A user interface 308, which may include an LCD screen and/or a keyboard, allows a user to interact with the ambulatory physiological monitoring unit 110. For example, the patient may start the real-time analysis when the patient experiences a particular symptom associated with an abnormal physiological condition (e.g., by selecting on option on a touch screen). The patient may also send a message to the remote monitoring system indicating that the patient has sensed a physiological event or that the patient has experienced a symptom associated with an abnormal physiological condition. Although the block depicting the user interface is shown as separate from the block depicting the ambulatory physiological monitoring unit, the user interface may be physically integrated into the ambulatory physiological monitoring unit.

The ambulatory physiological monitoring unit 110 can monitor a variety of physiologic parameters including blood pH, glucose, dissolved oxygen, carbon dioxide, breathing activity, heartbeat, ECG, and other parameters known in the art. The ambulatory physiological monitoring unit 110 can also monitor other ambulatory medical devices including an insulin pump or a pacemaker.

FIG. 3B is a block diagram of a remote monitoring center 120 according to embodiments of the present invention, which is configured to communicate with the ambulatory physiological monitoring unit 110 via a network. As shown in FIG. 3B, the remote monitoring center 120 can connect to a network through a wired connection 330. The remote monitoring center 120 can also connect to the network through a wireless connection (not shown). The remote monitoring center 120 includes a processor 325, a retrospective analysis module 327, and an evaluation module 329. The remote monitoring center 120 includes a receiver 323 that receives real-time analysis data through the wired connection 330. The remote monitoring center 120 can also be designed to receive raw physiological data through the wired connection 300 via a communications network. The ambulatory physiological monitoring unit 110 or removable memory of the ambulatory physiological monitoring unit 110 can also be mailed to the location where the remote monitoring center 120 is located so that raw physiological data can be uploaded to the remote monitoring center 120 through the wired connection 330.

After the remote monitoring center 120 receives the raw physiological data, the processor 325 executes a retrospective analysis module 327, which retrospectively analyzes the raw physiological data in detail. The retrospective analysis module 327 can analyze a portion of physiological data associated with a physiological event or all the physiological data that has been acquired during a monitoring session lasting a few minutes to several days. The retrospective analysis module 327 provides retrospective analysis data (e.g., information on the results of the retrospective analysis) to the evaluation module 329.

The evaluation module 329 evaluates the retrospective analysis data to determine the condition or status (physiological or non-physiological) of a patient. The evaluation module 329 can evaluate the retrospective analysis data against a set of predetermined rules to determine the physiological status of a patient. The evaluation module 329 can also transmit the retrospective analysis data or a summary or representation of the retrospective analysis data to a display of a user interface 320 so that a medical professional can review the retrospective analysis data, determine the status of the patient, and take appropriate action. The user interface 320 can be designed to accept commands or input from the medical professional. For example, the user interface 320 can include a menu of possible patient status levels that the medical professional can select based on her review of the retrospective analysis data. The user interface 320 can also include a menu of possible commands that the medical professional can select, such as a command to request additional physiological data or data regarding a different physiologic parameter from the ambulatory physiological monitoring unit 110.

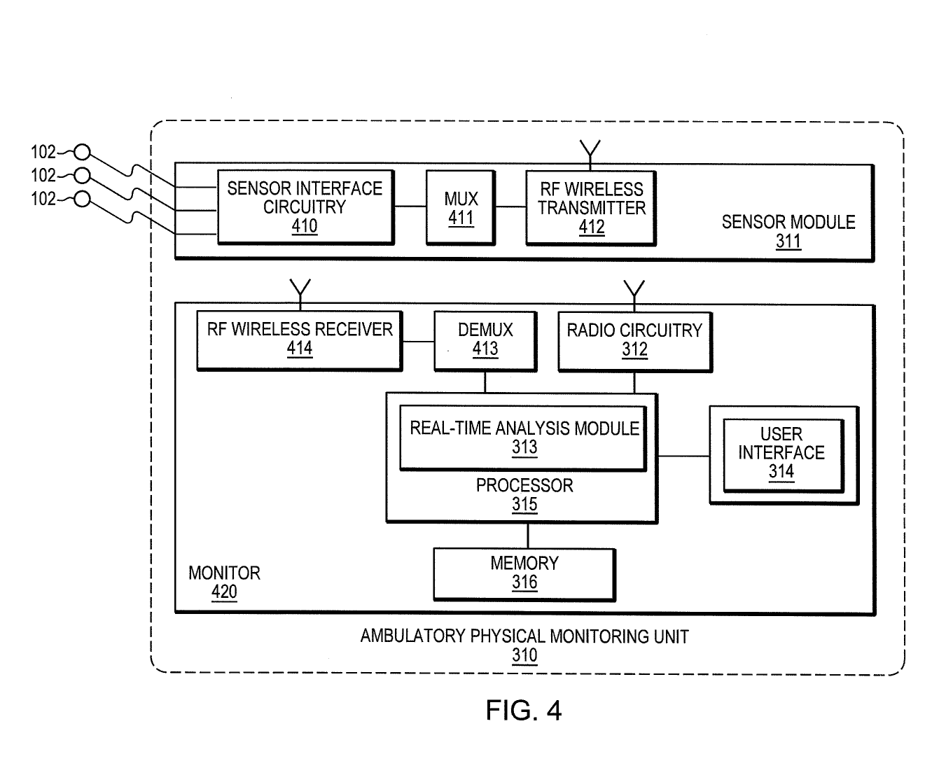

FIG. 4 is a block diagram of an embodiment in which a ambulatory physiological monitoring unit 310 is divided into two modules: a sensor module 311 and a monitor 420. The sensor module 311 includes electronic components that interface with and process the analog data acquired by the sensors 102. In particular, the sensor module 311 includes sensor interface circuitry 410, a multiplexer 411, and a radio frequency (RF) transmitter 412 (e.g., a Bluetooth® transmitter). The sensor interface circuitry 410 acquires analog physiological data from the sensors 102 and converts it into digital form. The multiplexer 411 takes the digital physiological data for each of the sensors 102 and combines it into a stream of digital physiological data. The RF transmitter 412 modulates a carrier signal with the stream of digital physiological data and transmits it to an associated RF receiver 414 (e.g., a Bluetooth® receiver) in the monitor 420.

The monitor 420 includes circuitry for analyzing, in real-time or near real-time, the digital physiological data transmitted from the sensor module 311. The monitor 420 can be any portable electronic device that is capable of wireless network communications, including a smartphone (e.g., a BlackBerry® device or an iPhone® device). The monitor 420 includes the RF receiver 414, a demultiplexer 413, radio circuitry 312, a processor 315 that executes the real-time analysis module 313, memory 316, and a user interface 314. The RF receiver 414 receives and demodulates the physiological data signal to obtain the digital physiological data and the demultiplexer 413 provides digital physiological data for each of the sensors 102 to the processor 315. The processor 315 executes the real-time analysis module 313, which, in one embodiment, analyzes a sliding window of digital data in a digital physiological data stream. The real-time analysis module 313 can include any known algorithms for interpreting digital physiological data, such as digital ECG data. The real-time analysis module 313 can generate real-time analysis data and transmit it to a display of the user interface 314. The processor 315 can also store the digital physiological data in memory 316.

The radio circuitry 312 can transmit real-time analysis data (e.g., information on the results of the real-time analysis of the digital physiological data stream, including whether a physiological event has been detected) and a portion of the digital physiological data stream associated with the real-time analysis data.

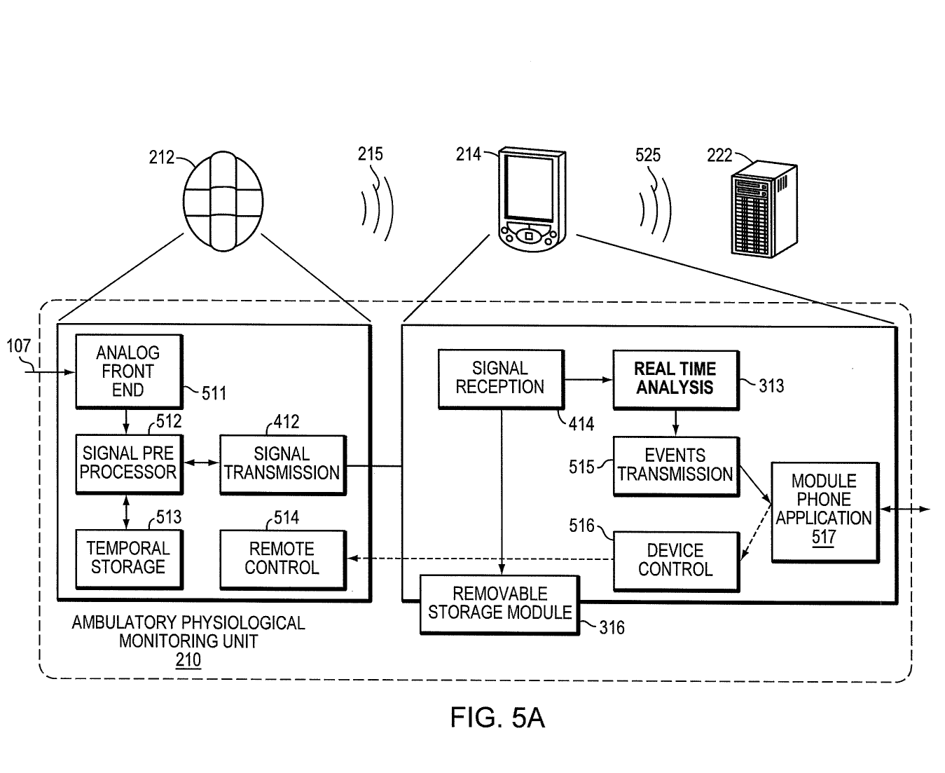

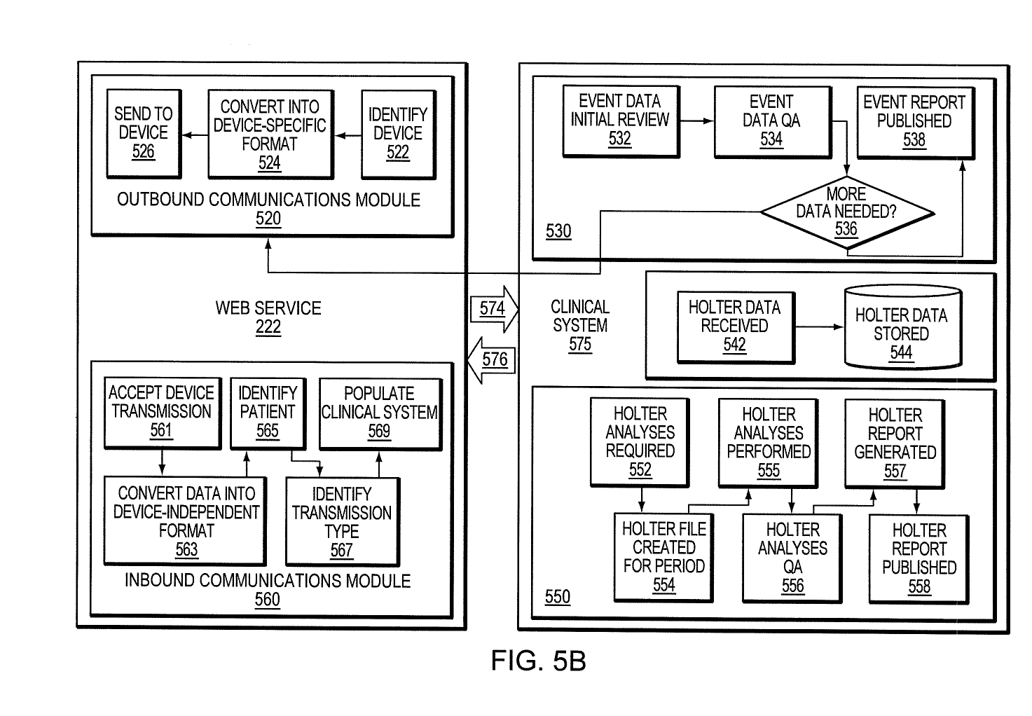

FIG. 5A is a functional block diagram of the ambulatory physiological monitoring unit 210, including the sensor module 212 and the monitor 214, and the web service computer 222 of FIG. 2. The sensor module 212 includes an analog front end 511 for acquiring analog ECG signals 107, a signal preprocessor 512 for converting the ECG signals into digital form, temporal storage 513, a signal transmission module 412, and a remote control module 514. The monitor 214 includes a signal reception module 414, a real-time analysis module 313, an events transmission module 515, a device control module 516, a removable storage module 316, and a mobile phone application 517.

The temporal storage module 513 of the sensor module 212 temporarily stores digital ECG data from the signal preprocessor 512 for subsequent transmission to the monitor 214. The signal transmission module 412 of the sensor module 212 transmits digital physiological data to the signal reception module 414 of the monitor 214 via an RF wireless network connection 215, (e.g., a Bluetooth® network connection). In this embodiment, the real-time analysis module 313 determines whether an event has occurred based on an analysis of the digital physiological data. If the real-time analysis module 313 determines that a predetermined event has occurred, the events transmission module 515 will transmit information regarding the event to the mobile phone application 517. The mobile phone application 517 then transmits the information regarding the event to a Web service 222, which, in turn, stores and distributes the information to appropriate computers or portable electronic devices connected to the Web service 222 via a network connection.

The mobile phone application 517 can also be configured to receive instructions or commands from the monitoring center 120 using generic communication protocols such as the hyper text transfer protocol (http) implemented on the Web service 222. The device control module 516 of the monitor 214 can interpret those instructions or commands and control the sensor module 212 accordingly. The device control module 516 can control the sensor module 212 by communicating with the remote control module 514 via the RF wireless network 215. For example, the mobile phone application 517 may receive a command from the monitoring center 120 via the Web service 222 to retransmit ECG data for a specified time period, to acquire and transmit data for a different physiologic parameter, or to reprogram or to change the parameters of the sensor module 212 or the monitor 214. In the case where the monitoring center 120 requests that the monitor 214 retransmit ECG data for a specified time period, the device control module 516 can initiate a process to resend the ECG data stored in the removable storage module 316. In the case where the monitoring center requests that the sensor module 212 acquire and transmit data for different physiologic parameters (e.g., pulse-oximetry data), the device control module 516 automatically configures the sensor module 212 to acquire data for the different physiologic parameters through the remote control 514. The device control module 516 can also request through the remote control module 514 that the signal transmission module 412 retransmit physiological data stored in temporal storage 513 to the monitor 214.

As shown in FIG. 5A, the monitor 214 of the ambulatory physiological monitoring unit 210 communicates with the web service computer 222 via a wireless communications link 525. FIG. 5B is a functional block diagram of the web service computer 222 and a clinical system computer 575 that is configured to communicate with the web service computer 222 over a communications network 574, 576. In some embodiments, the web service computer 222 serves as a communications interface between the monitor 214 and the clinical system computer 575. The web service computer 222 executes an inbound communications module 560 when it detects a transmission, such as a packet or series of packets, from the monitor 214. The inbound communications module 560 accepts the transmission 561 and converts the data contained in the transmission into a device-independent format 563. After the data has been converted, the patient associated with the monitoring unit 210 or other portable device is identified 565. Then, the transmission type is identified 567. The inbound communications module 560 then populates the clinical system computer 569 with the data contained in the received transmission. If the data contained in the received transmission is Holter data 542 or any other type of physiological data, this data is stored in a database 544 residing in the clinical system computer 575.

After Holter data is received and stored, the Holter analysis module 550 determines whether Holter analysis is required 552. In some instances, Holter analysis would not be required because the real-time analysis data is sufficient to conclusively determine the status of the patient. If Holter analysis is required, a Holter file is created for the period during which Holter data was collected 554. Next, a Holter analysis is performed on the Holter file 555. The results of the Holter analysis then undergo a quality assurance subroutine 556, which can include displaying the results of the Holter analysis on a computer screen so that a medical professional can review it. The quality assurance subroutine 556 can also include executing a test program, which performs an analysis of the results of the Holter analysis.

If the Holter analysis quality assurance routine determines that the results of the Holter analysis are accurate, a Holter report is generated 557 and published 558. The Holter report can include a summary of the Holter analysis results. The Holter report can also indicate the physiological status of the patient and suggest possible courses of action.

When the clinical system computer 575 receives event data, the event analysis module 530 performs an initial review of the event data 532 and executes a quality assurance subroutine 534 on the event data. If the event analysis module 530 determines that more data is needed 536 or that data for a different or additional physiologic parameter, an outbound communications module 520 identifies the device associated with the event data 522 (e.g., an ambulatory health monitor), converts a message requesting physiological data into a format that can be read by the identified device 524, and sends the message to the device 526. If no further data is needed, an event report is published 538.

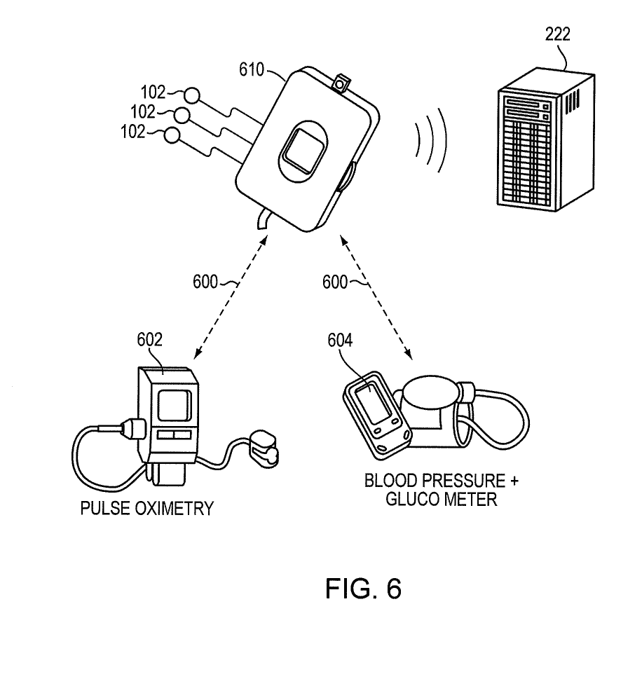

FIG. 6 is a diagram of an ambulatory health monitoring unit 610 that is configured to communicate 600 via a wireless network (e.g., a Bluetooth® network) with other medical components such as a pulse-oximetry measurement device 602 and a blood pressure and glucose meter 604. In this embodiment, physiological data from the pulse-oximetry measurement device 602 and a blood pressure and glucose meter 604 can be incorporated into real-time analysis of ECG data by the ambulatory physiological monitoring unit 610, which can act as a medical components communication hub.

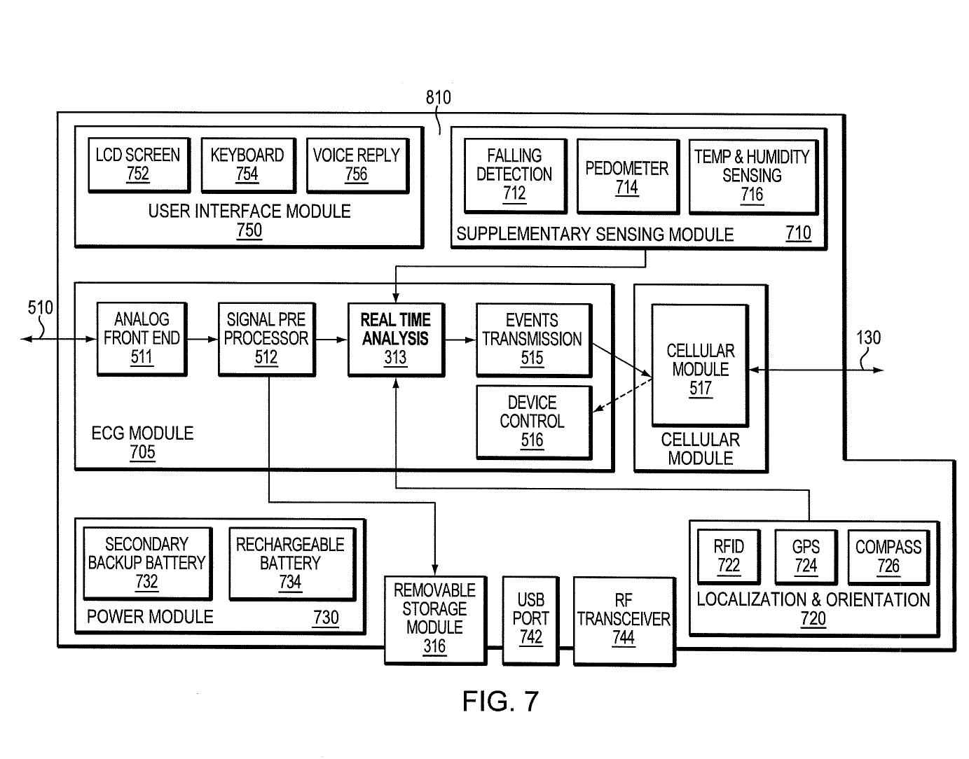

FIG. 7 is a block diagram of another embodiment of an ambulatory physiological monitoring unit 810. In this embodiment, many of the components of the sensor module 212 shown in FIG. 5 are incorporated into the ambulatory physiological monitoring unit 810 in the form of an ECG module 705. The ECG module 705, like the sensor module 212, includes an analog front end 511 that receives signals from ECG leads 510, a signal preprocessor 512, and real-time analysis module 313, an events transmission module 515, and a device control module 516. In some embodiments, the real-time analysis module 313 is a software module that performs an analysis of the physiological data and determines whether a physiological event has occurred. The ambulatory physiological monitoring unit 810 also includes various modules, which interact with the ECG module 705. These various modules can include a user interface module 750, a supplementary sensing module 710, a cellular module 517 for connecting with a communications network 140, a localization and orientation module 720, and a power module 730.

The user interface module 750 includes an LCD screen 752, a keyboard 754, and a voice reply module 756. The voice reply module can include an audio speaker, circuitry, and software, which generate voice prompts to the user. The user interface module 750 can also include a visual alert, such as a blinking LED, which can prompt the user of a problem or other event. The LCD screen 752 can allow the user to view information regarding the user's physiological condition and environment. The keyboard 754 allows the user to control some of the components and operation of the ambulatory physiological monitoring unit 810.

The ambulatory physiological monitoring unit 810 also includes a supplementary sensing module 710, which can sense non-physiologic parameters. For example, in this embodiment, the supplementary sensing module 710 includes a falling detector 712, which detects whether the patient has fallen down from a standing position, a pedometer 714, and a temperature & humidity sensor 716. The real-time analysis module can use data from these sensors to determine, for example, the probability that a physiological event has occurred.

The ambulatory physiological monitoring unit 810 also includes a localization and orientation module 720. The localization and orientation module can include an RFID tag 722 for identifying the ambulatory physiological monitoring unit or a person associated with it. The localization and orientation module 720 can also include a GPS 724 and a compass 726 for locating the patient. If a GPS signal is available, the localization and orientation module 720 can determine the location of the user using the GPS 724. If a GPS signal is unavailable (e.g., when the user enters a building), the localization and orientation module 720 can determine the user's location based on the last known location of the user stored in the GPS 724, the orientation of the user from the compass 726, and the position information from the pedometer 714.

The ambulatory physiological monitoring unit 810 also includes a USB port 742 and an RF transceiver 744 (e.g., a Bluetooth® transceiver) for wired and wireless communications with nearby electronic devices. The power module 730 includes a rechargeable battery 734 and a secondary backup battery 732 in case power supplied by the rechargeable battery 734 is disrupted.

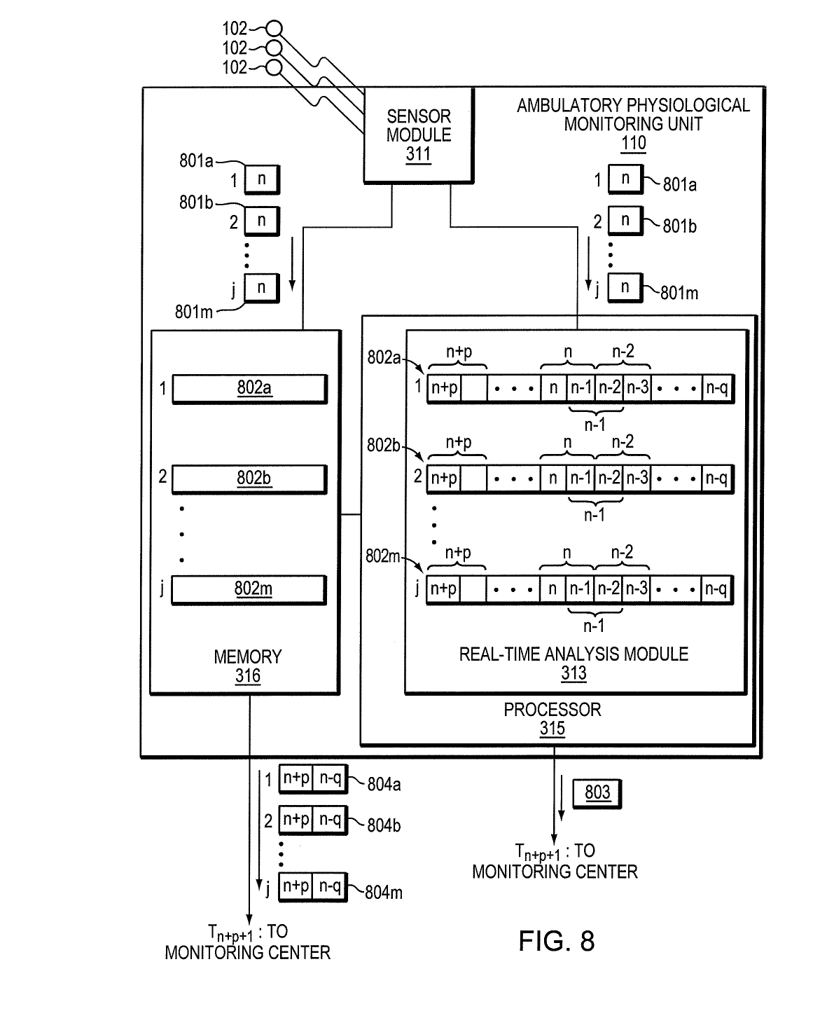

In some embodiments, when the real-time analysis module 313 detects an event from a segment of physiologic data, the ambulatory physiological monitoring unit 810 transmits information about the event and the physiological data that surrounds the event to the remote monitoring center for detailed retrospective analysis. FIG. 8 is a flow diagram illustrating this process. Sensor module 311 acquires analog physiological signals from multiple sensors 102 and provides a stream of digital physiological data 801a-m corresponding to each sensor 102 to the processor 315, which stores the digital physiological data 802a-m in memory 316 and executes a real-time analysis module 313, which analyzes the stream of digital physiological data 802a-m. The digital physiological data stored in memory 316 can be organized by sensor. For example, all physiological data corresponding to sensor 1 (802a) are aggregated in one location in memory 316 and all physiological data corresponding to sensor j (802m) are aggregated in a different location in memory 316.

The digital data stream 802 is also analyzed by the real-time analysis module 313. In one embodiment, the real-time analysis module 313 analyzes segments of the physiological data stream 801, 802 using a sliding window technique. According to the sliding window technique, the real-time analysis module 313 analyzes overlapping or adjacent segments or windows of the physiological data stream 802. For example, at time n−1, the real-time analysis module analyzes physiological data that has been acquired at both times n−1 and n−2 (i.e., the data in the window indicated by the bracket labeled n−1). At time n, the real-time analysis module analyzes physiological data that has been acquired at the current time n and the previous time n−1 (i.e., the data in the window indicated by the bracket labeled n).

After the real-time analysis module 313 completes its analysis, the processor generates and transmits a message 803. For example, at time n+p, the processor generates and transmits a message 803, which can contain real-time analysis data (e.g., information regarding the occurrence of an event) generated based on an analysis of physiological data acquired from at least one sensor at times n and n−1 (i.e., the data in the window at time n). At time n+p+1, the message 803 is transmitted to the monitoring center 120. At the same time, physiological data acquired at times n+p and n−q (804a-m) (i.e., the data segments coming before and after the segment of data acquired at time n), which is stored in memory 316, is transmitted to the monitoring center 120. The monitoring center 120 can analyze the physiological data acquired at times n+p and n−q to better understand an event detected at time n. The ambulatory physiological monitoring unit 110 can also transmit a large segment of raw physiological data that includes the data acquired at time n. For example, the ambulatory physiological monitoring unit 110 can transmit the raw physiological data acquired starting at time n−q and ending at time n+p.

The real-time analysis module 313 can perform any analysis on the physiological data that is known in the art. For example, the real-time analysis module 313 may be configured to analyze electrocardiography data. Also, the real-time analysis module 313 may be configured to analyze all types of physiological data, including electrocardiography data, pulse-oximetry data, blood pressure data, and temperature data.

At any particular time, physiological data associated with the real-time analysis data can be transmitted to the monitoring center. For example, the monitoring center 120 may request physiological data acquired by the sensor module 311 at times n and n−1. In other words, the monitoring center 120 may request the physiological data associated with the real-time analysis performed at time n. In response to this request, the physiological data acquired by the sensor module 311 at times n and n−1 is transmitted to the monitoring center 120.

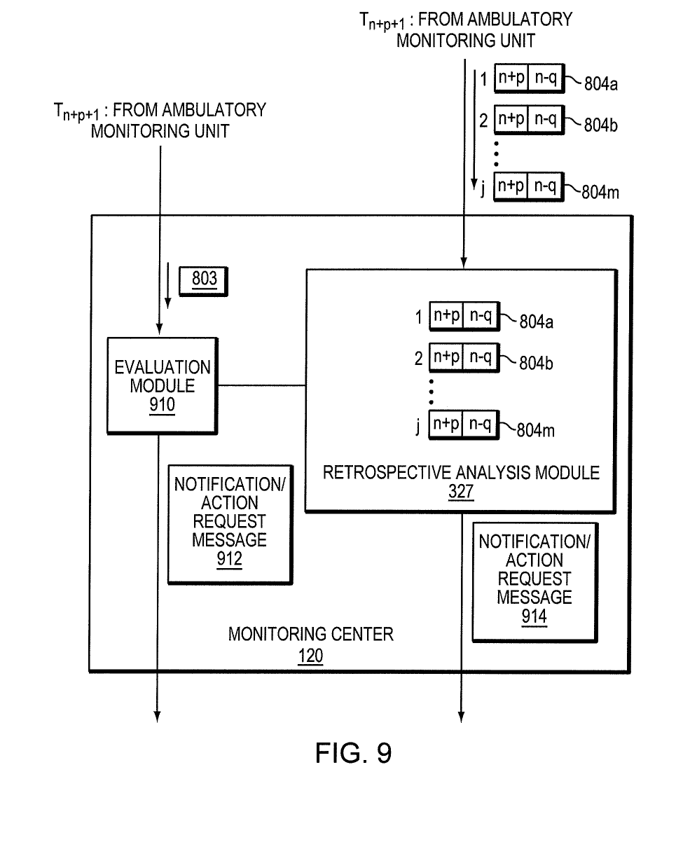

As illustrated in FIG. 9, the message 803 generated by the real-time analysis module 313 of the ambulatory physiological monitoring unit 110, which can contain information regarding an event that has been detected, is evaluated by an evaluation module 910 of the monitoring center 120. The evaluation module 910 can format and display the event information so that it can be reviewed and evaluated by a medical professional. If the medical professional needs more detailed and specific information to diagnose the patient and determine an appropriate course of treatment, the medical professional can quickly request and receive detailed retrospective analysis data. The medical professional does not have to wait to receive the physiological data at the end of a monitoring session.

The evaluation module 910 can also automatically evaluate the information contained in the message 803 and determine a course of action based on the information contained in the message 803. For example, the evaluation module 910 can include a series of rules, which automatically determines a diagnosis and takes appropriate action. If the information in the message 803 indicates a life-threatening event, the evaluation module can automatically generate and transmit a message (e.g., the notification/action request message 912) to paramedics or a care-giver requesting that they immediately go to the patient's location. If the information in the message 803 is insufficient to determine an accurate and/or conclusive diagnosis for the patient, the evaluation module can evaluate the more detailed retrospective analysis data to determine a diagnosis.

The retrospective analysis module 327 in the monitoring center 120 may then analyze the physiological data to determine, among other things, whether the information contained in the real-time analysis data message 803 reconciles with the physiological data. The retrospective analysis module 327 can also correlate physiological data acquired from different sensors 102.

In some instances, the physiological data transmitted to the monitoring center 120 may include gaps or other anomalies. For example, the physiological data may include a gap because the user removes the sensors from his body to take a shower or the ambulatory physiological monitoring unit turns off (e.g., because the rechargeable battery fails). If the ambulatory physiological monitoring unit is turned on, the retrospective analysis module 327 can determine whether the sensors are connected to the user's body or to the ambulatory physiological monitoring unit 110 by monitoring for a signal pattern transmitted from the ambulatory physiological monitoring unit 110, which indicates that the sensors are disconnected. The ambulatory physiological monitoring unit 110 can determine whether the sensors are properly connected by performing an impedance check.

If the retrospective analysis module 327 finds gaps or anomalies in the physiological data, it can compensate for the gaps or anomalies and re-construct the time line using known techniques in the art. For example, the retrospective analysis module 327 can use other physiological or non-physiological data that corresponds to a time period within the gap. The monitoring center 120 can also request that the ambulatory physiological monitoring unit 110 acquire and analyze additional physiological data from the ambulatory physiological monitoring unit 110 so that the retrospective analysis module 327 has sufficient physiological data to perform an accurate and complete retrospective analysis.

The retrospective analysis module 327 can generate a notification/action request message 914 based on the results of the retrospective analysis module and transmit it to an appropriate device or network-connected computer associated with a medical professional or another appropriate person. For example, if the retrospective analysis module 327 determines that its analysis does not reconcile with the information contained in real-time analysis data message 803, it can generate a message 914 notifying a medical professional that the retrospective analysis data does not reconcile with the information contained in real-time analysis data message 803.

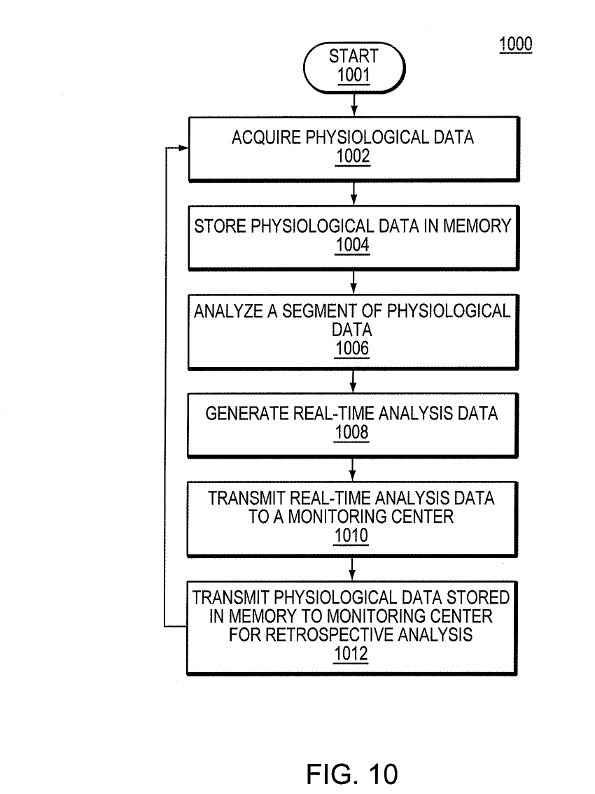

FIG. 10 is a flow diagram illustrating a process 1000 that can be executed in the ambulatory physiological monitoring unit 110. After starting the process 1001, physiological data is acquired from sensors attached to a patient's body 1002. The physiological data is then stored in memory 1004 and analyzed 1006. Based on an analysis of the physiological data, real-time analysis data is generated 1008 and transmitted to a monitoring center 1010. After real-time analysis data is transmitted to the monitoring center, physiological data associated with the real-time analysis data and stored in memory is transmitted to the monitoring center for detailed retrospective analysis 1012 and the process 1000 returns to step 1002.

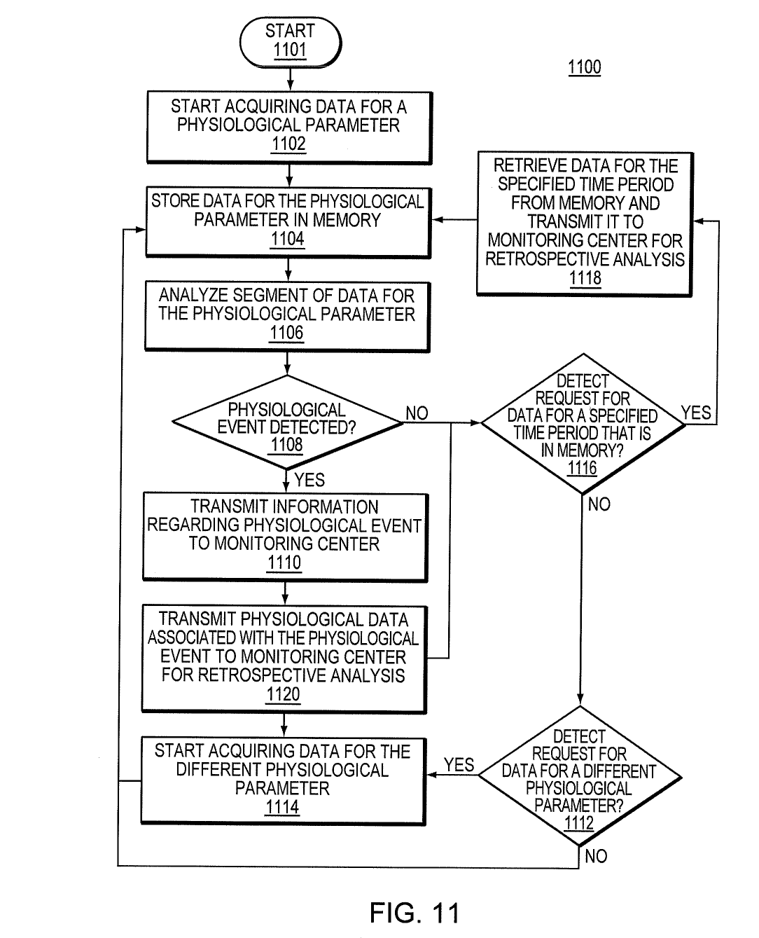

The ambulatory physiological monitoring unit 110 can acquire different types of physiological data or can retrieve physiological data stored in memory in response to a request from the monitoring center 120. As illustrated in FIG. 11, the ambulatory physiological monitoring unit can start 1101a process 1100 that continuously acquires data for a physiologic parameter 1102 and stores that data in memory 1104. In step 1106, windows or segments of data for the physiologic parameter are analyzed. A module in the ambulatory physiological monitoring unit 110 then determines whether a physiological event has been detected based on the analysis 1108. If a physiological event has been detected, information regarding the physiological event and physiological data associated with the physiological event are transmitted to the monitoring center 1110, 1120.

If a physiological event has not been detected or after physiological data associated with the physiological event is transmitted to the monitoring center, a module in the ambulatory physiological monitoring unit 110 determines whether a request for data for a specified time period that is stored in memory has been requested by the monitoring center 120 (step 1116). If such a request is detected, the ambulatory physiological monitoring unit 110 retrieves the data for the specified time period from memory and transmits it to the monitoring center for retrospective analysis 1118. If a request for data a for a specified time period is not detected, a module in the ambulatory physiological monitoring unit 110 determines whether a request for data for a different physiologic parameter has been received from the monitoring center 120 (step 1112). If a request for data for a different physiologic parameter is detected, the ambulatory physiological monitoring unit starts acquiring this data 1114 and the process 1100 returns to step 1104. Otherwise, the process 1100 returns to step 1104.

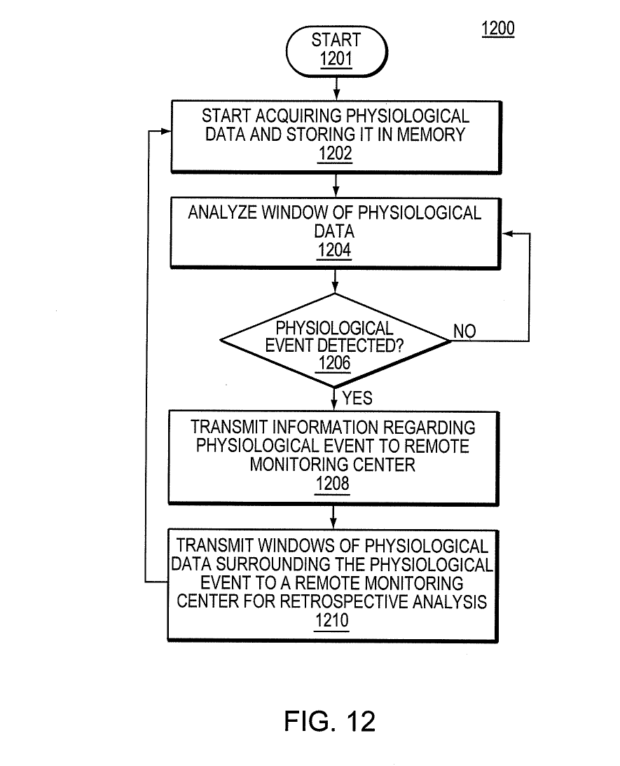

FIG. 12 is a flowchart illustrating process steps that are executed by the processor of the ambulatory physiological monitoring unit according to another embodiment of the invention. After starting 1201, physiological data (e.g., ECG data) is acquired and stored in memory 1202. In step 1204, a window of physiological data is analyzed. If, as a result of the analysis of the physiological data, a physiological event is detected 1206, information regarding the event and windows of physiological data surrounding the event are transmitted to a remote monitoring center 1208, 1210. If a physiological event is not detected, the process 1200 returns to step 1204 and process steps 1204-1210 are repeated for another window of physiological data.

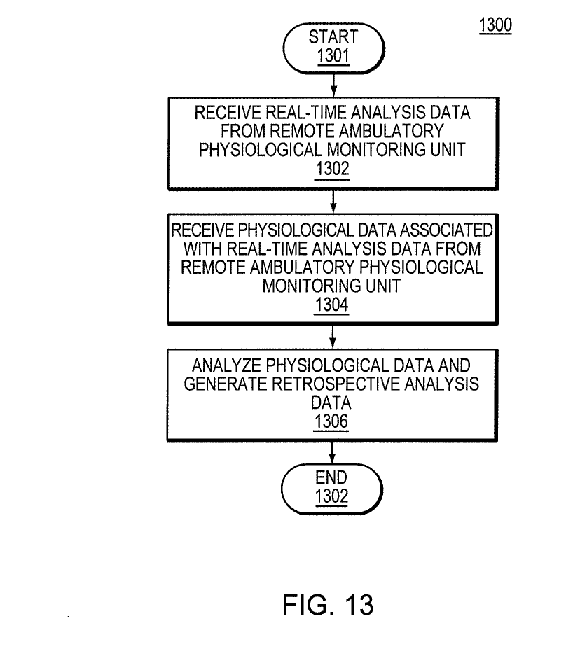

FIG. 13 is a flowchart illustrating the steps performed by the monitoring center 120. After starting 1301, real-time analysis data is received from an ambulatory physiological monitoring unit 1302. At this point, the real-time analysis data can be evaluated to determine the physiological status of the patient and a message notifying a medical professional of the patient's physiological status can be generated and sent to the appropriate portable electronic device or computer. In step 1304, physiological data associated with the real-time analysis data is received, and, in step 1306, the physiological data is analyzed to generate retrospective analysis data. In some embodiments, the physiological data associated with the real-time data is received in response to a request for the physiological data from the monitoring center 120.

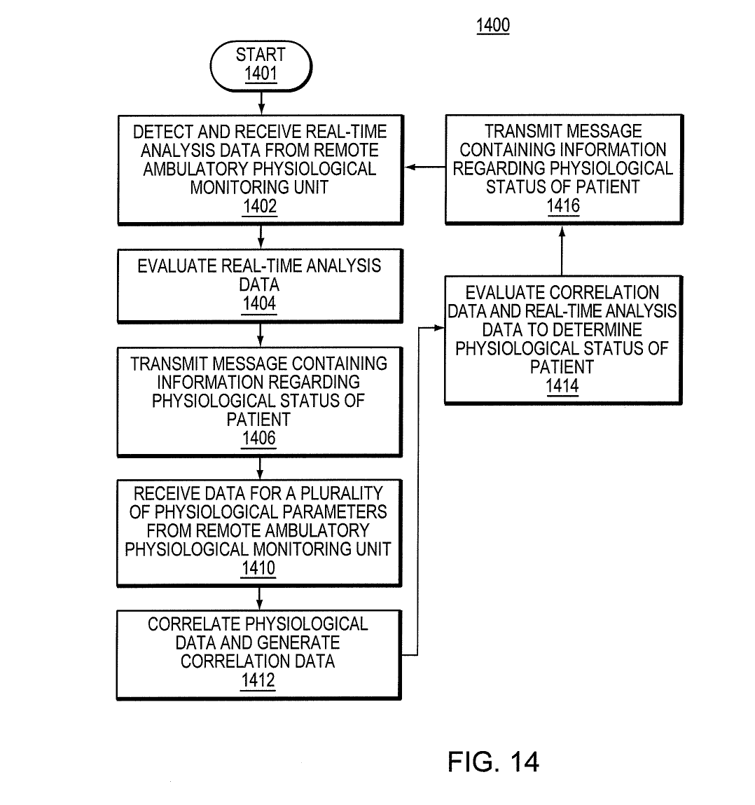

The remote monitoring center can automatically evaluate the real-time analysis data and the retrospective analysis data and notify a medical professional of the physiological status of the patient or provide a medical professional with a summary of the retrospective analysis data. FIG. 14 is a flowchart illustrating such a process. After the process 1400 starts 1401, real-time analysis data or physiological data is detected and received from an ambulatory physiological monitoring unit 110 (1402). In step 1404, the real-time analysis data is evaluated. Based on the evaluation, a message containing information regarding the physiological status of the patient can be generated and transmitted to an appropriate computer or portable electronic device 1406 (e.g., a medical professional's BlackBerry® device or personal computer). In some embodiments, evaluating the real-time analysis data 1404 and transmitting a message 1406 can include displaying the real-time analysis data (e.g., information regarding the occurrence of an event) on a computer screen.

Next, data for a plurality of physiologic parameters is then received from the remote ambulatory physiological monitoring unit 1410. The data for a plurality of physiologic parameters is correlated and correlation data is generated 1412. In step 1414, the correlation data and the real-time analysis data are evaluated to determine the physiological status of the patient. A message containing information regarding the physiological status of the patient can then be transmitted to a networked device that is accessible by a medical professional or other relevant care-giver 1416 and the monitoring center can continue to detect and receive real-time analysis data.

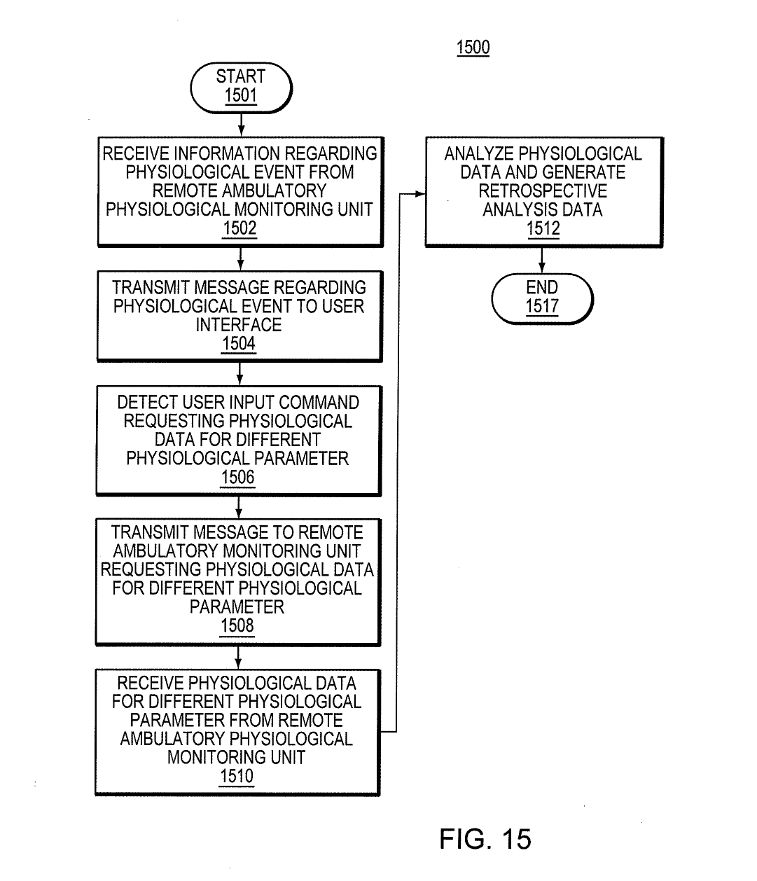

In some instances, a medical professional may determine that the physiological data may be insufficient (e.g., because it contains gaps or invalid data) for an accurate and specific retrospective analysis. The remote monitoring center 120 can allow a medical professional to request real-time analysis data or physiological data from the ambulatory physiological monitoring unit 110. FIG. 15 is a flowchart illustrating a process that allows a medical professional to request additional physiological data or data for a different physiologic parameter. After the process starts 1501, information regarding a physiological event is received from a remote ambulatory physiological monitoring unit 1502. A message regarding that physiological event is transmitted to a user interface 1504 at the remote center 120 so that a medical professional can determine an appropriate course of action. If the medical professional determines that data for a different physiologic parameter is needed, she can input an appropriate command to the user interface requesting data for a different physiologic parameter. In step 1506, a user input command requesting data for a different physiologic parameter is detected and, in step 1508, a message requesting data for a different physiologic parameter is generated and transmitted to the ambulatory physiological monitoring unit 110. In step 1510, physiological data for different physiological parameter is received from an ambulatory physiological monitoring unit 110. Before ending 1517, the physiological data is analyzed and retrospective analysis data is generated 1512.

The above-described systems, modules, and methods can be implemented in digital electronic circuitry, in computer hardware, firmware, and/or software. The implementation can be a computer program product. For example, the implementation can be in a machine-readable storage device, for execution by, or to control the operation of, data processing apparatus. The implementation can, for example, be a programmable processor, a computer, and/or multiple computers.

A computer program can be written in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Method steps can be performed by one or more programmable processors executing a computer program to perform functions of the invention by operating on input data and generating output. Method steps can also be performed by and an apparatus can be implemented as special purpose logic circuitry. The circuitry can, for example, be a FPGA (field programmable gate array) and/or an ASIC (application specific integrated circuit). Modules, subroutines, and software agents can refer to portions of the computer program, the processor, the special circuitry, software, and/or hardware that implement that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer can include, can be operatively coupled to receive data from, and/or can transfer data to one or more storage devices for storing data (e.g., magnetic, magneto-optical disks, or optical disks).

Data transmission and instructions can also occur over a communications network. Information carriers suitable for embodying computer program instructions and data include all forms of non-volatile memory, including by way of example semiconductor memory devices. The information carriers can, for example, be EPROM, EEPROM, flash memory devices, magnetic disks, internal hard disks, removable disks, magneto-optical disks, CD-ROM, and/or DVD-ROM disks. The processor and the memory can be supplemented by, and/or incorporated in special purpose logic circuitry.

To provide for interaction with a user, the above described techniques can be implemented on a computer having a display device. The display device can, for example, be a cathode ray tube (CRT) and/or a liquid crystal display (LCD) monitor. The interaction with a user can, for example, be a display of information to the user and a keyboard and a pointing device (e.g., a mouse or a trackball) by which the user can provide input to the computer (e.g., interact with a user interface element). Other kinds of devices can be used to provide for interaction with a user. Other devices can, for example, be feedback provided to the user in any form of sensory feedback (e.g., visual feedback, auditory feedback, or tactile feedback). Input from the user can, for example, be received in any form, including acoustic, speech, and/or tactile input.

The above described techniques can be implemented in a distributed computing system that includes a back-end component. The back-end component can, for example, be a data server, a middleware component, and/or an application server. The above described techniques can be implemented in a distributed computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communications network). Examples of communications networks include a local area network (LAN), a wide area network (WAN), the Internet, wired networks, and/or wireless networks.

The system can include clients and servers. A client and a server are generally remote from each other and typically interact through a communications network. The relationship of client and server arises by virtue of computer programs running on the respective computers and having a client-server relationship to each other.

Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), 802.11 network, 802.16 network, general packet radio service (GPRS) network, HiperLAN, evolution-data optimized (EVDO) network, long term evolution (LTE) network), and/or other packet-based networks. Circuit-based networks can include, for example, the public switched telephone network (PSTN), a private branch exchange (PBX), a wireless network (e.g., RAN, Bluetooth® (Personal Area Network (PAN)), code-division multiple access (CDMA) network, time division multiple access (TDMA) network, global system for mobile communications (GSM) network), and/or other circuit-based networks.

The transmitting device can include, for example, a computer, a computer with a browser device, a telephone, an IP phone, a mobile device (e.g., cellular phone, personal digital assistant (PDA) device, laptop computer, electronic mail device), and/or other communication devices. The browser device includes, for example, a computer (e.g., desktop computer, laptop computer) with a world wide web browser (e.g., Microsoft® Internet Explorer® available from Microsoft Corporation, Mozilla® Firefox available from Mozilla Corporation). The mobile computing device includes, for example, a BlackBerry® device.

Certain embodiments of the present invention were described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. A method for monitoring a patient, the method comprising:
executing at least one data acquisition module in an ambulatory physiological monitoring unit to acquire physiological data;
executing a real-time analysis module in the ambulatory physiological monitoring unit, the real-time analysis module analyzing segments of physiological data and generating real-time analysis data for each segment;
transmitting the real-time analysis data to a remote monitoring center; and
transmitting physiological data associated with the real-time analysis data to the remote monitoring center for further analysis.

2. The method of claim 1, wherein analyzing segments of physiological data comprises monitoring for a physiological condition or event and the real-time analysis data comprises information regarding the physiological condition or event, which is generated in response to detecting the physiological condition or event.

3. The method of claim 2, wherein monitoring for a physiological condition or event comprises determining whether the physiological data or processed physiological data reaches at least one predetermined level.

4. The method of claim 1, wherein the physiological data is electrocardiography data.

5. The method of claim 1, wherein the physiological data comprises physiological data for a plurality of physiologic parameters of at least one body system.

6. The method of claim 5, wherein the physiological data for the plurality of physiologic parameters of at least one body system comprises pulse-oximetry data.

7. The method of claim 1, wherein the ambulatory physiological monitoring unit transmits the real-time analysis data to the remote monitoring center at predetermined intervals.

8. The method of claim 1, wherein transmitting the physiological data to the remote monitoring center comprises storing the physiological data in a memory module of the ambulatory physiological monitoring unit and uploading the physiological data from the memory module to the remote monitoring center.

9. The method of claim 8, wherein the data acquisition module is in wireless communications with the real-time analysis module and the memory module.

10. The method of claim 1, further comprising:
detecting a message requesting physiological data or real-time analysis data for at least one physiologic parameter; and
executing the data acquisition module and real-time analysis module to acquire and to analyze physiological data for the at least one physiologic parameter.

11. The method of claim 10, wherein the message is generated and transmitted automatically by the ambulatory physiological monitoring unit, generated and transmitted by a user interface associated with the ambulatory physiological monitoring unit, or both.

12. The method of claim 10, wherein the message is generated and transmitted by the remote monitoring center, generated and transmitted by a service in communication with ambulatory physiological monitoring unit, or both.

13. A method for monitoring a patient, the method comprising:
receiving real-time analysis data from a remote ambulatory physiological monitoring unit, the real-time analysis data being based on an analysis of segments of physiological data acquired by the remote ambulatory physiological unit;
receiving physiological data associated with the real-time analysis data from the remote ambulatory physiological monitoring unit;
executing a retrospective analysis module to generate retrospective analysis data based on the physiological data.

14. The method of claim 13, wherein the real-time analysis data is information regarding a physiological event or condition detected by the remote ambulatory physiological monitoring unit.

15. The method of claim 13, wherein the physiological data comprises a plurality of physiologic parameters associated with the same and/or different body systems, and wherein executing the retrospective analysis module comprises correlating physiological data for the plurality of physiologic parameters.

16. The method of claim 13, wherein receiving real-time analysis data and physiological data from a remote ambulatory physiological monitoring unit comprises receiving the real-time analysis data via a wireless communications link and uploading the physiological data from a memory module on the remote ambulatory physiological monitoring unit via a wired communications link

17. The method of claim 13, further comprising executing an evaluation module for evaluating the real-time analysis data or retrospective analysis data to determine the physiological status of the patient.

18. The method of claim 17, wherein executing an evaluation module comprises executing a user interface module for allowing user interaction with the real-time analysis data and retrospective analysis data.

19. The method of claim 18, further comprising transmitting a message to the remote ambulatory monitoring unit requesting physiological data or real-time analysis data in response to the user interface module detecting a user input command requesting physiological data or real-time analysis data.

20. The method of claim 17, wherein evaluating the real-time analysis data or retrospective analysis data comprises determining whether the physiological data or real-time analysis data is sufficient to generate conclusive diagnostic information.

21. The method of claim 17, wherein the message comprises a request for additional physiological data.

22. The method of claim 17, wherein the message comprises a request for physiological data for a different physiologic parameter.

23. The method of claim 17, further comprising transmitting a message containing information regarding the physiological status of the patient.

24. An ambulatory physiological monitoring unit, comprising:
a physiological data acquisition module for acquiring physiological data;
a real-time analysis module for analyzing segments of the physiological data and generating real-time analysis data;
a storage module for storing the physiological data; and
a transceiver configured to transmit the real-time analysis data to a remote monitoring center via a communications network in response to at least one event or physiological condition detected by the real-time analysis module, the transceiver further configured to transmit physiological data associated with the at least one event or physiological condition to the remote monitoring center for retrospective analysis of the physiological data.

25. The ambulatory physiological monitoring unit of claim 24, further comprising:
a user interface module configured to accept input from a user regarding at least one event or physiological condition, the user interface module further configured to generate a message containing information regarding the event or physiological condition, the transceiver further configured to transmit the message to the remote monitoring center.

26. A system for monitoring a patient, the system comprising:
an ambulatory physiological monitoring unit, comprising:
a physiological data acquisition module for acquiring physiological data;
a real-time analysis module for analyzing segments of the physiological data and for generating real-time analysis data for each segment of physiological data;
a storage module for storing the physiological data; and
a transceiver for transmitting the real-time analysis data and physiological data associated with the real-time analysis data via a communications network; and
a remote monitoring center in network communications with the ambulatory physiological monitoring unit, the remote monitoring center comprising:
a transceiver for receiving the real-time analysis data and the physiological data from the ambulatory physiological monitoring unit and for transmitting messages requesting physiological data to the ambulatory physiological monitoring unit via the communications network;
a retrospective analysis module for performing a retrospective analysis based on the physiological data and generating retrospective analysis data; and
an evaluation module for evaluating the physiological status of the patient based on the real-time analysis data or the retrospective analysis data.
